(19) United States
(12) Patent Application Publication
(10) Pub. No.: US 2008/0193927 A1
(43) Pub. Date: Aug. 14, 2008

(54) Method for Determining the Abundance of Sequences in a Sample

(76) Inventors: Wofgang Mann, Neudrossenfeld (DE); Christoph Gauer, Munchen (DE)

Correspondence Address: DILWORTH & BARRESE, LLP, 333 EARLE OVINGTON BLVD., SUITE 702, UNIONDALE, NY, 11553, US

(21) Appl. No.: 11/631,986

(22) PCT Filed: Jul. 27, 2005
(86) PCT No.: PCT/EP2005/008156

§ 371 Date: Jan. 9, 2007

(30) Foreign Application Priority Data
Jul. 27, 2004 (DE) 10 2004 03 285.8

(51) Int. Cl. C12Q 1/68; C12M 1/34

(52) U.S. Cl. 435/6; 435/287.2

(57) ABSTRACT

The invention relates to a method for determining the abundance of a given sequence or several sequences identical or nearly identical to the given sequence in a sample. The method comprises the following steps: carrying out one or more amplification reactions by means of which several different sections of the sequence or sequences of the sample can be amplified to give an amplified product, detection of whether given different sections of the sequence in the sample have been amplified and determination of the number of the sequence(s) in the sample by means of the abundance of the presence or otherwise of the given different sections in the amplified product.

The present invention relates to a method for determining the abundance of a given sequence or several sequences identical or nearly identical (homologous) to the given sequence in a sample.

### BACKGROUND TO THE INVENTION

Apart from sequence analysis, the quantitative analysis of nucleic acids is one of the most important challenges in molecular medicine. A basic understanding of the biology of cells, tissue and organisms requires a knowledge of the composition and abundance of genetic sequences, e.g. at DNA level, and their transcripts (RNA level). Individual differences between organisms and causes of genetic illnesses and predispositions lie in the differences between sequences (mutations, e.g. deletions, insertions) and the abundance with which the sequences occur. As a result, quantitative analyses of the genome (DNA) and the transcriptome (RNA) have become the main issues of molecular medicine.

The totality of an organism's genetic information is anchored in its genome. Changes in the information carrier (genetic sequences of nucleic acids with the base sequences of G, A, T and C;=DNA) may manifest themselves as illness. In many cases, a quantitative statement concerning defined sequence sections is required for diagnostic purposes. There are various examples of clinical profiles that are attributable to different abundances of genetic sequences:

### Trisomy/Monosomy of Full Chromosomes

Trisomy 21, Down syndrome: a full chromosome (21) is affected and occurs with 3 copies per cell (rather than 2 copies).

### Repeat Motive

Huntington's disease: a given motive (CAG) occurs in direct succession in over 37 copies. The predisposition to developing the illness increases with the number of repeats of this motive. Other examples of unstable trinucleotide sequences in individuals are Kennedy's syndrome or spinocerebral ataxia 1.

### Chromosomal Microdeletions of Small Sequence Sections

It has emerged that chromosomal microdeletions play a part in a growing number of clinical syndromes. There are numerous examples, such as Wolf Hirschhorn syndrome (deletion 4p16.3), Williams Beuren syndrome (7q11.23, involves the deletion of an entire gene) or also Prader-Labhart-Willi syndrome (15q11-q13), in which only the paternal genes are affected. Less common are microduplications, although locating such sections is very difficult today for methodological reasons.

### Point Mutations

Many clinical profiles emerge because precisely one base position is changed, which has an adverse effect on the function of the resulting protein. For these cases, too, (single nucleotide polymorphisms, SNP's) a quantitative statement is of crucial importance, because the mutations or alleles do not occur in all cells or can be expressed with differing abundance. Mutations of this type, which are not present in gene sequences, very frequently occur in the genome and do not usually lead to a clinical profile. However, they are suitable as markers, because many tumor cells tend to lose one of the two parental alleles (loss of heterozygosity, LOH). The observation that of two original sequence variants only one is still present is of great potential significance in tumor diagnosis. One of the methodological developments used to detect this state reliably and quantitatively is digital PCR (U.S. Pat. No. 6,440,706 B1).

In all the aforementioned examples, molecular diagnosis involves the quantification of sequence sections, i.e. the frequency with which a given sequence is contained in a sample must be detected.

### STATE OF THE ART

The following methods are mainly used in today's research and diagnosis, in order to solve the objects described above for DNA.

Chromosome-specific probe molecules for in situ hybridization using the FISH method (fluorescence in situ hybridization) are known from U.S. Pat. No. 5,817,462. This involves various combinations of different fluorophors being used to detect all human chromosomes simultaneously.

The chromosomes to be analysed are brought into contact with paint-marked hybridization probes, so that sequence-complementary sections can be found. Following sequence-specific hybridization there is a washing stage, after which the cell's fluorescence signals are evaluated under a fluorescence microscope. If a fluorescence signal exists, the sequence also exists. The presence of a complete chromosome, for example, can thereby be concluded. If no fluorescence signal exists, either the chromosome is not present or there is a microdeletion in the area of the probes. Today FISH can be used for the parallel determination of the copy number of several different sequences within a genome, which are distinguished in the evaluation by the fluorescence paint used. The number is limited by the number of fluorescence paints used simultaneously. Typically, cell populations, which all have the same genetic status, are used.

A FISH analysis is very hard to validate. DE Rooney ed., 2001: Human Cytogenetics Constitutional Analysis, Oxford University Press, states the following in relation to the interpretation of the results of a FISH analysis: “Probes used for interphase analysis should be chosen to hybridize with high efficiency (>90%)”. This statement means, firstly, that at least 100 cells have to be counted and, secondly, it generally precludes individual cell diagnostics using FISH. This method is not adequate for individual cell diagnostics.

Another approach in which the number of copies of several sequences can be determined in parallel is CGH (comparative genomic hybridization, WO 00/24925, Karyotyping Means and Methods). In this case, a patient's DNA is marked using a fluorescent dye (e.g. red), a reference DNA with a second dye (e.g. green). The same amounts of the different DNA populations are mixed and hybridized on a glass surface with a chromosome spread. Complementary strands will compete for the bonding sites on the chromosome sections. If the sequence sections are equally abundant in patient and reference DNA, a ratio of 1:1 emerges between green and red. If one colour predominates, this indicates either a duplication or deletion of the corresponding sections in the patient DNA. The chromosome spread is analysed in the fluorescence microscope, which limits the resolution of the method, it is in the region of 10-30 Mb (1 Mb=1 megabase=10⁶ sequence building blocks). During the CGH of a chromosome spread, only precisely one probe (marked red or green) can be bonded to a defined sequence in a single chromosome. Only the poor spatial resolution of the method results in several signals being received side by side, which then statistically allow a ratio analysis.

A particular embodiment of the method is matrix CGH (chip or array format), in which rather than a chromosome spread, the gene sections are present in the form of discrete measuring points of a DNA array. Here, too, a comparison is made between the intensities of two hybridization signals. For CGH, the sample must either be amplified (e.g. by PCR) or there must be a large number of nominally identical cells present.

The quantitative real-time PCR method is suitable in principle for detecting the smallest quantities of nucleic acids (in principle, a copy of a sequence). The quantitative analysis is guaranteed by means of internal standards (Hagen-Mann, K & Mann, W. (1995): RT-PCR and alternative methods to PCR for in vitro amplification of nucleic acids. Exp. Clin. Endocrinol. 103: 150-155). The method is used for routine diagnostics. However, the amount of starting material cannot be randomly reduced, since with a small number of start molecules (10-100) as the starting material, the stochastic error due to the exponential amplification is very large, thereby precluding a quantitative statement.

Apart from PCR, there are other enzyme-based amplification methods, which do not permit a quantitative statement in the aforementioned area (e.g. NASBA, LCR, SDA RT-PCR or Qβ replicase; Overview in Hagen-Mann & Mann 1995).

All the aforementioned methods have various disadvantages in the quantitative analysis of sequences, which makes them unsuitable for an absolute statement in relation to copy numbers.

There exists today no simple, reliable method of counting sequence sections (in the range of 0, 1, 2, 3 to roughly 10), because two developments run completely counter to one another:
a) work is carried out without amplification, which means that a large number of cells is needed (a figure of 10⁶ would be typical for CGH); the fluorescence cannot be measured otherwise. Due to the complexity of the hybridization reaction (non-specific links, cross-reactions, slow and mostly unknown kinetics) and costly sample preparation (sample purification, unknown efficiency with the integration of fluorescence dyes), the quantification of gene sequences by experimentation is highly complex and the interpretation of the results in now way trivial;
b) a quantitative amplification reaction is carried out with a small amount of starting material, in order to determine the copy number of a defined sequence (as with 0, 1, 2, 3 . . . ), e.g. from the signal increase with a real-time PCR. In this case, the error will be high, due to the exponential amplification rate.

A method of determining the relative abundance of sequences in a sample is known from U.S. Pat. No. 6,440,706 B1 and is referred to as digital amplification or digital PCR. This involves the sample being diluted and distributed between a large number of reaction vessels, so that a reaction vessel should, if possible, contain no more than a single molecule of one of the sequences being investigated. The sample divided between several reaction vessels is then amplified with several primers, each primer being specific to one of the sequences and provided with a marker. Following the amplification, the markers incorporated in the amplified product are used to identify which of the sequences was present in which reaction vessel. By counting the reaction vessels, each of which contains a particular sequence, the quantity ratio of the sequences in the original sample can be determined. This method brings with it considerable uncertainties, which is essentially due to the dilution series, as it can never be determined with absolute certainty whether a reaction vessel actually contains several sequence molecules, causing the result to be distorted. In addition, this method can only be used to determine relative, rather than absolute, quantity ratios.

Another method enabling the relative abundance of sequences to be determined is known from WO 2004/027089. This method is intended to determine, for example, whether one of several definable subsets (i.e. separate nucleic acids or sequences) of a genetic material occurs more or less abundantly in a sample than the other definable subsets. A concrete embodiment of this method from the state of the art relates to the determination of the relative abundance of individual chromosomes in a cell, e.g. in order to determine whether aneuploidy exists. In this embodiment of the method disclosed in WO 2004/027089, a single cell amplification, e.g. a whole genome amplification (WGA), is first carried out with a non-specific primer or several such primers, wherein several target sequences specific to a chromosome had to be theoretically amplified in each case. However, because this sort of whole genome amplification is not 100% efficient, not all the target sequences that could theoretically be amplified by the primers are actually amplified in the statistical mean. Following the amplification reaction, specific target sequences are detected for each chromosome.

It is not possible with any of the methods described above to count a number of, e.g. ten or fewer essentially identical sequences in a sample. Most methods are unsuitable in principle for the quantitative detection of such a small number of sequences. Only using digital PCR can the relative abundances of different sequences, which are present in relatively small quantities, be detected. On account of a dilution series being used, determining the relative abundance of sequences, which are only present in a very small number of 10 or fewer, for example, is problematic.

The invention is based on the object of creating a method for determining the abundance of a given sequence or sequences identical or nearly identical (homologous) to the given sequence in a sample, which can be carried out simply, cheaply and reliably, even for a small number of sequences.

The object of the invention is solved according to claim 1. Advantageous embodiments of the invention are indicated in the dependent claims.

The inventive method for determining the abundance of a given sequence or identical or nearly identical (homologous) sequences in a sample comprises the following steps:
carrying out one or more amplification reactions with which several target sections of the sequence or sequences in the sample can be amplified into an amplified product;
detecting whether given target sections of the sequence in the sample have been amplified and
determining the abundance of the sequence(s) in the sample based on the abundance of the presence or otherwise of given target sections in the amplified product.

A preferred embodiment of the inventive method is indicated in claim 2. This method of determining the abundance n of a given sequence or several sequences identical or nearly identical to the given sequence in a sample comprises the following steps:
(a) providing a sample containing the sequence in an abundance n to be determined;
(b) providing primers with which a number m of target sections of the given sequence in the sample can be amplified into different amplified products in each case;
(c) carrying out one or more amplification reactions using the sample from (a) and the primers from (b), in which the reaction conditions are chosen such that the number of successful amplification reactions depends on the abundance n of the given sequence in the sample;
(d) detecting the amplified target sections from step (c) and determining the number of successful amplification reactions;
(e) determining the abundance n of the given sequence contained in the sample.

The abundance n of the given sequences in the sample will preferably be determined by comparison with one or several controls, in which a known abundance of the given sequence is present. The controls can either be control samples that have undergone the method in the invention in parallel, in which case the same reaction conditions are used for the parallel control samples as for the amplification reaction(s) with the sample. It is also possible to subject the control samples to a procedure according to the invention, irrespective of the sample, and produce validated data or reference data, which serve as controls for comparison with the sample.

Several target sections of the original sequences may, for instance, be amplified by using several primer pairs or primer combinations, each of which are specific to a given target section or a small number of given target sections in the sequence or through the use of primers, which are each specific to a large number of given target sections.

For the purposes of the present invention, the term primer not only covers individual primers, but also primer pairs (i.e. one forward and one backward primer) and primer combinations (more than one forward and backward primer for a given target section).

PCR methods that use a large number of given target section-amplifying primers are referred to as IRS-PCR (Inter-Repetitive Sequence PCR) or WGA-PCR (Whole Genome Amplification PCR), i.e. the primers are non-specific insofar as they amplify a large number of different sequences.

The inventors of the present invention have established that with the amplification of several different given target sections of a sequence, the number of different amplified target sections depends on the number of the sequence originally present in the sample. The smaller the number of the sequence present in the sample, the smaller the number of different target sections amplified by it too.

It is assumed that the reason for this is that the success of each amplification is associated with a given probability or efficiency, i.e. that each amplification is not carried out with absolute certainty. So, for instance, with simultaneous amplification of several different sections, competition emerges between the amplification reactions of the different individual sections, so that if only one or a small number of given sequences are present in the sample material, fewer of the different individual sections are amplified than if a large number of sequences were to be amplified.

The efficiency depends on a large number of factors, such as the choice of primer, the length of the sequence to be amplified and the other reaction conditions, such as, with a PCR, the temperature protocol, cycle duration, cycle number, react and concentration, reaction volume, polymerases, etc. The person skilled in the art is able to adjust these parameters so that a desired efficiency is achieved.

By establishing these factors, it is also possible to determine the efficiency of an amplification in a given range. If, for instance, a sequence is to be amplified with the greatest possible certainty, but is only present in small numbers, it is advantageous for the efficiency to be as close as possible to 1. This is advantageous, for example, for diagnostic PCR (pathogen detection) and other similar applications.

If, on the other hand, as in the method in the present invention, a distinction is also to be made as to whether a sample contains a given sequence only once, twice, three times or in a similarly small number, it is more advantageous to set the efficiency of the amplification at an average value, e.g. in the region of 0.1 to 0.9, preferably 0.2 to 0.8, preferably 0.3 to 0.7, preferably 0.4 to 0.6 and, most preferably, at around 0.5. The amplification efficiency should lie within a range that allows a statistically significant statement to be made in relation to the absolute quantity of sequences originally present, i.e. nucleic acid molecules.

The notion of efficiency is therefore interpreted in the following such that it indicates the amplification probability on the assumption that any amount of starting material is present. The efficiency of an amplification suitable for the invention typically lies in the range 0.5 to 1. On the other hand, the actual amplification probability of a given section with a small number of start copies of the sequence depends on the amount of starting material, i.e. the number of given sequences in the sample, and may in principle cover the entire possible range from 0 to 1.

For example, it is the case that under given conditions with the amplification of several target sections of a sequence, even when suitable primers are selected for the several target sections concerned, it is not possible to amplify all target sections in each amplification reaction. This applies, in particular, when the original templates, i.e. the original sequence, from which the target sections are to be amplified, is only present in small copy numbers, e.g. in the region of 0, 1, 2, 3, 4, 5, 6, 7, 8, 9, 10.

Since each amplification reaction has a certain error probability attached to it, it is very difficult with state-of-the-art methods to distinguish between different samples containing different numbers of templates for the amplification reactions. For example, a sample may contain two sequences, from which given target sections are to be amplified in each case, and a second sample may contain three of these sequences, from which particular target sections are to be amplified. It has not been possible to date, using state-of-the-art amplification methods, to conclude the number of original templates, i.e. the abundance of sequences originally present in a sample, if the numbers lie within this small range.

However, the method in the invention makes it possible to determine the absolute number of sequences originally present in a sample, particularly when this number, which is referred to as n for the purposes of the present invention, lies within the range n=0, 1, 2, 3, 4, 5, 6, 7, 8, 9, 10. n should preferably lie within the range 0-100, preferably 0-30, preferably 0-10, preferably 0-5. It is particularly preferable for the method in the invention to be set so that samples with sequence numbers n=0, 1, 2, 3 and 4 can be quantitatively determined.

To achieve this, the amplification conditions are preferably selected such that the efficiency of the amplification reaction for n=1 lies in the range 0.2-0.8, preferably 0.4-0.6. It is particularly preferable for the amplification efficiency to be in the region of 0.5. This means that if a sequence whose number n in the sample=1 (i.e. this sequence occurs in the sample a single time, so that only a single template is available) and a number m of target sections is to be amplified for this sequence, based on an efficiency of 0.5 in the statistical mean, only half the target sections can be detected following the amplification reaction.

The method contained in the present invention thereby enables a quantitative statement to be made on the copy number of the sequence present in a sample. For this purpose, amplification reactions are carried out, in order to amplify several different target sections in the sequence into an amplified product. If the given sequence from which the several target sections are to be amplified is only present in a very small copy number and the amplification efficiency is less than 1, it is highly probable that not all the chosen target sections will actually be amplified in the amplification reaction, even if this is carried out over several cycles. If the result of a sample amplification reaction is compared with a given sequence by detecting the several different target sections and if this result is compared with an amplification reaction on a sample containing 2 copies of the given sequence conducted under the same conditions, there is a statistically significant probability that a higher number of positively detectable amplified target sections will be obtained for the sample with 2 copies.

This statistical approach is explained in greater detail below.

The presence or otherwise of an amplified product based on a defined PCR protocol (including temperature protocol, cycle number, react and concentration, volume, threshold value for detection of the amplified product) conducted using a specific primer or primer pair depends on the copy number of the sequence in the starting material. The counting method according to the invention is explained below by way of example in a thought experiment.

The copy numbers 0, 1 and >=2 in a sample are to be differentiated with a certainty of >=90%. If these copy numbers are referred to a chromosome in a pole body, cases of monosomy (copy number 2 in the pole body), healthy cell (copy number 1 in the pole body) and trisomy (copy number 0 in the pole body) are differentiated. First of all, PCR's are carried out on control samples with a known copy number n=0, 1, 2 with m=8 different fluorescence-marked primers and a defined PCR protocol. The amplified product is detected by hybridization on an array with a threshold value for the presence of an amplified product 5-times the background signal. Where k=100 experiments the following abundance distribution is obtained for the three cases of copy numbers n:

TABLE 1
Number of positive amplifications

| | 0/8 | 1/8 | 2/8 | 3/8 | 4/8 | 5/8 | 6/8 | 7/8 | 8/8 |
|---|---|---|---|---|---|---|---|---|---|
| n = 0 | 98 | 2 | 0 | 0 | 0 | 0 | 0 | 0 | 0 |
| n = 1 | 2 | 13 | 24 | 57 | 3 | 1 | 0 | 0 | 0 |
| n = 2 | 0 | 0 | 0 | 0 | 3 | 40 | 35 | 20 | 2 |

Assuming that the distribution remains the same for larger numbers k, the following conclusions can be drawn from the table: If the same experiment is carried out on a sample with an unknown copy number and the results ⅝, 6/8, ⅞ or 8/8 positive are obtained, the copy number n=2 can be inferred with the required certainty. If the result is 0/8 positive, there can be >90% confidence that the original copy number was n=0. If the result is 2/8, ⅜, the copy number n=1 existed with the required certainty. The results ⅛ and 4/8 cannot be decided with the required confidence. If these cases are also to be decided with the required certainty, the number of different amplifications m can be increased, for example, or the PCR protocol changed. In a further thought experiment, m=12 and the following Table 2 abundance distribution is obtained with the corresponding control samples:

TABLE 2
Number of positive amplifications

| | 0/12 | 1/12 | 2/12 | 3/12 | 4/12 | 5/12 | 6/12 | 7/12 | 8/12 | 9/12 | 10/12 | 11/12 | 12/12 |
|---|---|---|---|---|---|---|---|---|---|---|---|---|---|
| n = 0 | 95 | 5 | 0 | 0 | 0 | 0 | 0 | 0 | 0 | 0 | 0 | 0 | 0 |
| n = 1 | 0 | 0 | 3 | 20 | 44 | 30 | 3 | 0 | 0 | 0 | 0 | 0 | 0 |
| n = 2 | 0 | 0 | 0 | 0 | 0 | 0 | 0 | 3 | 27 | 45 | 15 | 7 | 3 |

Here, there is no overlap on the number of positive amplification reactions, i.e. all relevant values n can be differentiated in the required confidence interval. Another thought experiment on the control samples is now carried out under PCR conditions with the following result (reduction in the cycle number from I=30 to I=27):

TABLE 3
Number of positive amplifications

| | 0/12 | 1/12 | 2/12 | 3/12 | 4/12 | 5/12 | 6/12 | 7/12 | 8/12 | 9/12 | 10/12 | 11/12 | 12/12 |
|---|---|---|---|---|---|---|---|---|---|---|---|---|---|
| n = 0 | 98 | 2 | 0 | 0 | 0 | 0 | 0 | 0 | 0 | 0 | 0 | 0 | 0 |
| n = 1 | 0 | 1 | 14 | 22 | 40 | 20 | 3 | 0 | 0 | 0 | 0 | 0 | 0 |
| n = 2 | 0 | 0 | 0 | 0 | 0 | 0 | 3 | 10 | 30 | 32 | 15 | 7 | 3 |

Here, a clear statement can be made for the values 1/12 and 6/12. In a further iteration step it is possible to try, e.g. by adjusting the threshold detection value, once again to obtain results based on the same experiments that do not overlap.

The general aim of optimising the counting method in accordance with the invention is to obtain a reliable differentiation of the copy numbers n in the desired range with as few PCR reactions as possible. To achieve this, the PCR conditions and, if necessary, the primers must be optimised accordingly. The parameters obtained may then be added to the kit in the invention and applied by the user to interpret the results.

Table 4 shows how the statistical certainty of the statement rises if the number of independently investigated results is increased. A Gaussian distribution is taken as the basis:

TABLE 4

| | | | Difference |
|---|---|---|---|
| | | | probability |
| | Result | Result | between two |
| | copies | copies | experiments |
| Number of | target | target | t-test |
| target | sequence = 1 | sequence = 2 | assuming |
| sections | Positive | Positive | different |
| investigated m | reactions/m | reactions/m | variances |
| 8 | 4/8 | 6/8 | 0.335 |
| 16 | 8/16 | 12/16 | 0.154 |
| 32 | 16/32 | 24/32 | 0.039 |

It is unimportant whether the results in relation to the target sequences are obtained in several individual experiments with nominally identical starting material (for the last case in 32 individual reactions independent of one another) or in a single multiplex reaction (e.g. a 32-plex based on a single cell).

When implementing the method in accordance with the invention, it is possible that in multiplex experiments there will be correlations between the amplification reactions.

For the method according to the invention, it is preferable for the reaction conditions to be set such that the distributions of successful amplification reactions described above by way of example are as sharp as possible. This means, particularly for the detection of given sequences with low abundances, that samples where n=1 can be differentiated from those where n=2 and those where n=3.

Surprisingly, it has emerged that with amplification reactions, particularly PCR amplifications, the distributions, as described above, with the corresponding conditions set, are far sharper than one would assume.

The method according to the invention is therefore also particularly suited to determining the abundance of a small number of sequences, the number of which preferably lies within the range 0 to 10. Depending on the number range of the number of sequences to be expected, the number of independent PCR reactions m and the PCR conditions may preferably be set such that the reliability of the result can be optimised according to the number of sequences to be expected.

The sample material is preferably made up of the genome of only one single cell, whereby the method according to the invention can be used to reliably determine whether the given sequence is not present or is present once or twice or three times or four times or five times, etc.

The method according to the invention is also particularly suited to pole body analysis.

The given sequence may be a chromosome, for example, but it may also be a fragment of a chromosome or part of a chromosome. However, the given sequence may also be a gene or a larger sequence section. The method according to the invention may, in principle, be used for every type of nucleic acid and nucleic acid sequences, e.g. also for plasmids and other artificial sequences. The embodiments described below by way of example should not therefore be restrictively interpreted, but are suitable for any type of quantitative detection of nucleic acid sequences in a small number in the starting sample. The nucleic acid sequence may be a DNA, RNA, mRNA, cDNA or genomic DNA.

In particular, the method according to the invention is suitable for determining the number of chromosomes in a single cell, i.e. the method is suitable for determining the presence of aneuploidies.

The method according to the invention may be implemented in several embodiments.

In a first embodiment, which is described for a chromosome analysis, for example, a single cell amplification is carried out using specific primers. This does not require the cell to undergo WGA, i.e. non-specific amplification, first.

In a preferred second embodiment, however, WGA is carried out first, in order to amplify the nucleic acid material of a single cell or a few cells non-specifically. The specific amplification is then carried out in accordance with claim 1. With these two successive amplification reactions, e.g. PCR's, the number of amplified products depends on the copy number of sequences to be counted originally present in the sample. Tables are then determined by way of experiment in a similar way to Tables 1 to 3 for the entire process and on this basis the user can infer the copy number for his samples. Here, too, there is a certain probability distribution for the presence or otherwise of the amplified products.

With the specific amplification, the cell with specific primers or corresponding primer pairs/primer combinations undergoes an amplification reaction, in which the primers or primer pairs are chosen such that for each chromosome a number m of given and specific target sections can be amplified. For a chromosome analysis, the number m of the target sections to be amplified is preferably at least 4, more preferably at least 6, more preferably at least 8. More target sections may also be selected per chromosome, e.g. 10, 12, 14, 16, 20, 30 or even more. In this case, however, it is left to the person skilled in the art to select a suitable number of target sequences per chromosome. The number m of specific target sequences for each chromosome (wherein m is to be understood as being per chromosome) should be large enough, so that with a statistical distribution of successful and unsuccessful amplification reactions a statement can be made at the end on the numbers of template molecules originally present. On the other hand, the numbers of specific target sections per chromosome m should not be too high, so that the number of primers or primer pairs used does not exceed a reasonable number. As already mentioned, the person skilled in the art can himself choose the number m of target sections to be amplified per chromosome, depending on the analysis and amplification method, and also specify the corresponding amplification conditions.

For the purposes of the present invention, control experiments are carried out, in which the control samples are each chosen from one cell with a known number n of nucleic acid molecules, e.g. a cell in which the nucleic acid is completely absent, as control 0, a cell in which the nucleic acid is present, as control 1, etc. Next, all these control samples undergo an amplification reaction with appropriately selected primers or primer pairs and the amplification conditions are optimised such that for the control sample in which the nucleic acid occurs once (n=1), the number of specific target sections for the nucleic acid is in the range m=4 to 30 and the amplification efficiency is around 0.5. This means that if the control sample 2, which contains the nucleic acid in two copies, is amplified under the same amplification conditions, the amplification efficiency is significantly higher. This can also be seen from Table 1.

This first embodiment of the present invention is particularly well suited to detecting chromosome numbers in individual cells. The method is particularly suited to pole body analysis, in which a pole body can possess a haploid or diploid chromosome set. In this case, preferably around 4-30, preferably 6, preferably 8 target sections will be chosen per chromosome, which are specific to the respective chromosome. Each of the target sections preferably occurs only once on each chromosome and is thereby specific. If such a cell is now amplified with a primer set for a chromosome or also with several sets of such primers for all chromosomes, the amplification reactions, as mentioned above, do not yield an amplification product in all cases, i.e. some of the m target sections per chromosome are not amplified and are not detectable later either. For instance, the conditions can be set in such a way that if a chromosome is only present once, only four (as a statistical mean) of the eight target sections, for instance, of the respective chromosome can be amplified. As a result, this means that a subsequent detection with the corresponding probes yields a result of 4/8. If the amplification efficiency has been set at a value of 0.5 beforehand using control samples, it can be concluded from the result of 4/8 that the chromosome was simply present in the sample.

Example 1 can be used to investigate whether a pole body contains chromosome 2 once or twice. The question of whether chromosomes are present in a pole body once or twice is of significance to the investigation of pole bodies. In other questions, however, it may be wise to determine whether a given sequence occurs with a different abundance and whether the possible number range not only comprises two figures, as in this example with 1 and 2, but a number range of, for example, three, four or five figures. The method in the invention may also be used, in principle, in order to capture greater number ranges, e.g. whether a given sequence is contained in a sample three, four, five or six times. In this case, more different sections of the given sequence must be amplified and detected.

The method in the invention is particularly suited to determining abundance or to counting a small number of a given sequence, which, for instance, is smaller than 20, smaller than 10, preferably smaller than 5 or 3, since the statistical spread of the number of successfully amplified sequence sections is particularly marked with a small number with given sequences in the sample.

In example 1, the χ² test has been used as the statistical method. However, other statistical methods are also suitable for evaluation of the amplification results, such as, for example, the mean comparison (t test, F test), variance-analytical methods (ANOVA, MANOVA), multi-field χ² tests or hierarchical log-linear methods.

In a preferred second embodiment, it is possible, however, rather than subjecting the sample along with specific primers for the specific target sections to an amplification reaction, to conduct a whole genome amplification first. For a WGA amplification followed by a specific amplification in accordance with claim 1, it is also possible using a statistical determination of control samples with known numbers n of starting nucleic acids (templates) to establish the amplification conditions such that on account of the statistical distribution of positive, i.e. successful, and negative, i.e. unsuccessful, amplification reactions, the number (n) of nucleic acids originally present can be concluded from the number of amplified target sections compared with the number (m) of previously selected target sections. The abundance tables (Tables 1, 2, 3) then relate to the combination of the two amplifications.

One example of this second embodiment is given in example 1.

In the context of the invention, each amplification method, with which several different given sections of a sequence for detection are detected, is suited to amplifying the sample. An individual primer, as in example 1, may be used for this. However, it is also possible for several primer pairs to be used, each of which is specific to a given section or a small number of sections.

The amplified products may, e.g. be analysed by means of electrophoresis, hybridization analysis on a DNA array, a bead system or another optical measurement, electrical measurement or electrochemical measurement.

To determine the abundance of a given sequence in the genome of a single cell or a small number of nominally identical cells, the following variants of the method are advisable:
1. 1 cell, WGA (non-specific single cell amplification), spatial division of the following PCR reactions (marker PCR), detection of sections (corresponds to the above embodiment);
2. 1 cell, WGA (non-specific single cell amplification), detection of sections by complex hybridization;
3. 1 cell, multiplex PCR, direct detection of sections without further amplification;
4. small number of nominally identical cells, multiplex PCR with one cell per reaction vessel, detection of sections without further amplification;
5. small number of nominally identical cells, specific PCR (precisely one) reaction each with one cell per reaction vessel, detection of sections without further amplification;
6. small number of nominally identical cells, specific PCR (precisely one) reaction each with one cell per reaction vessel, amplification each with a different primer pair per reaction and cell, detection of sections.

Methods 1-3 may also be carried out with a small number of nominally identical cells; the number of which is preferably known, e.g. ≦10.

With variants 1 and 2 indicated above, a WGA is carried out, which corresponds to the WGA of the embodiment described above. This sort of single cell amplification is also referred to as statistical amplification.

In variant 2, the sections are detected without further amplification through complex hybridization. Complex hybridization refers to a method in which several probes are present simultaneously, as is the case with DNA arrays or bead systems.

With variants 3 and 4, a multiplex PCR is carried out. This is a PCR with several specific primer pairs, which are carried out simultaneously in a reaction vessel. With each primer pair, precisely one section of the sequence is preferably amplified. With this sort of multiplex PCR, two to ten sections can, advantageously, be amplified simultaneously. Problems emerge with a larger number of sections, because the amplifications then become too non-specific.

In variant 4, information on the genetic material of a few nominally identical cells is summarised in a sample. In variants 5 and 6, the genetic material of a small number of nominally identical cells is firstly investigated independently of one another in different reaction vessels with a specific PCR that amplifies precisely one section. This is followed by detection of the sections without further amplification (variant 5) or with further amplification (variant 6).

If several cells are used in the analysis, the uncertainty for determining the abundance grows. The optimum determination of abundance comes with the analysis of single cells. If several nominally identical cells are present, the results can be compared. The method in the invention is particularly suited to analysing the genome of a single cell (e.g. a pole body, individual foetal cells from the maternal blood, etc.).

The method in the invention can be used to determine the abundance of a given sequence in a sample. The given sequence may be present several times in separate molecules in the sample. However, it may also be formed several times in a strand. The method in the invention can therefore count a given sequence that occurs several times in a strand in the same way as a given sequence that is present in the form of separate molecules. The sequence to be determined must simply be long enough for several sections to be amplified independently of one another. The length of the given sequence is at least 100 bases, preferably a few 100 bases.

With the method according to the invention, the abundances of several different given sequences can be determined simultaneously, whereby here too the different sequences can be formed on different strands or on the same strand. The different sequences can also overlap on the same strand.

Using the method in the invention, relative abundances of a given sequence of different samples can be determined. However, the inventive method can also be validated by a series of tests, such that the abundance of the existence or otherwise of the given sections in the amplified product permits a statement in relation to the absolute number of given sequences in a sample.

The method in the invention may be used to determine the abundance of sequences located on a common strand and also to determine the abundance of sequences located on different strands. The sequences should simply be of sufficient length for different sections to be addressable by means of primers.

A further object of the invention is a kit for carrying out the method in the invention, comprising
(i) one or more specific primers with which a number m of target sections of the given sequence, the abundance of which is to be determined in a sample, can each be amplified into different amplified products
(ii) if appropriate, control samples for each possible value n for the abundance of the given sequence in the control sample and/or
(iii) if appropriate, results of amplification reactions with the primers from (i) and/or of control samples with the abundance of the sequence to be counted known, e.g. the control samples from (ii),
(iv) details of the reaction conditions for the amplification reactions.

The kit may further comprise one or more non-specific primers, with which the given sequence can be amplified non-specifically following a given protocol.

The results of the control amplification reactions may be indicated in the form of stored data or printed material may be included in the kit, from which the user can read out the results and compare them with his own results from genuine samples.

The method in the invention may also be carried out in a small space, e.g. on a solid carrier, chip or a slide or similar. The method may also be carried out on multiwell plates, e.g. microtitre plates. The solid carrier is preferably a slide, a CD or some other solid carrier used for DNA array formats.

A further object of the invention is an apparatus suitable for conducting the method according to the invention. The apparatus preferably contains a mechanism for detecting nucleic acids, which were “captured” using marked probes, for instance, i.e. a mechanism for detecting fluorescence or calorimetric measuring methods, for example. The mechanism further comprises either stored data, which serves as comparative data, enabling the results of the amplifications to be assigned to the control data, such that by means of the comparison, the absolute number of given sequences originally contained in a sample can be determined. Rather than control data, which is stored in the apparatus, it is also possible for the apparatus to contain, in addition or as an alternative to this, control positioning sites, on which control samples can be analysed under the same conditions as the samples. The control samples contain, e.g. the nucleic acid to be detected in the genuine experiment or else the given sequence in the following numbers: n=0, n=1, n=2, n=3, etc. The method according to the invention is then carried out with the controls in exactly the same way as with the genuine samples.

The invention is further explained with reference to the attached drawings in which:

FIG. 1 shows the results of a first example in a table and

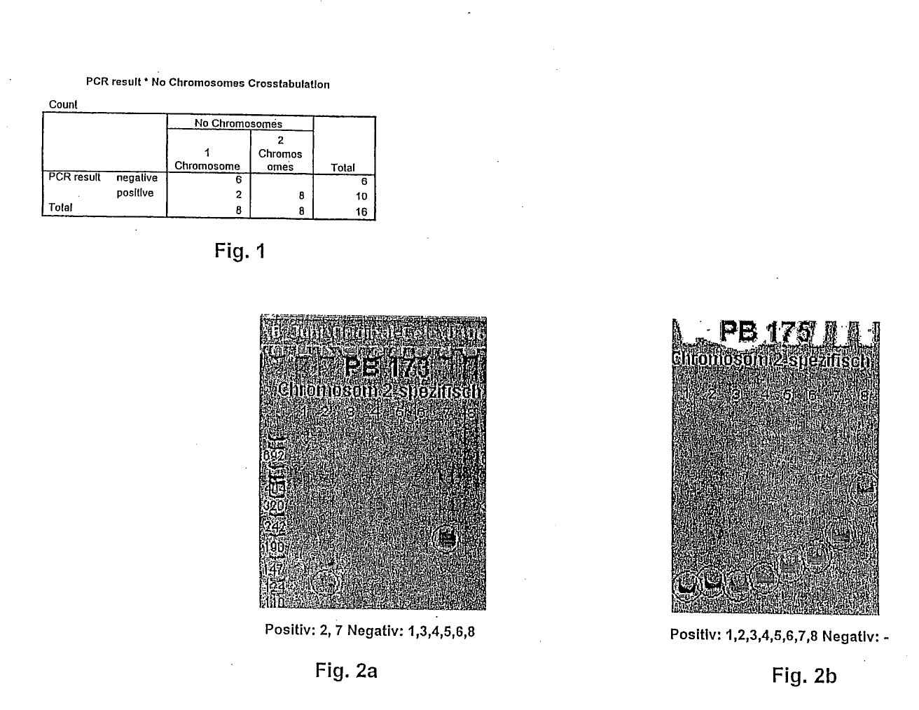

FIGS. 2a, 2b shows illustrations of an electrophoresis investigation for detecting the given sections.

FIG. 3 shows a comparison in relation to the existence or non-existence of chromosomes for 7 cell lines from the company Coriell, on the one hand, and in accordance with the method in the invention, on the other. Clear box: Chromosomes present (results of the methods agree). Hatched box: Chromosomes not present (results of the methods agree). Dotted box: Method according to the invention shows the presence of a chromosome, the other methods do not. Black box: Method in the invention shows the non-existence of the chromosome; the other methods its existence.

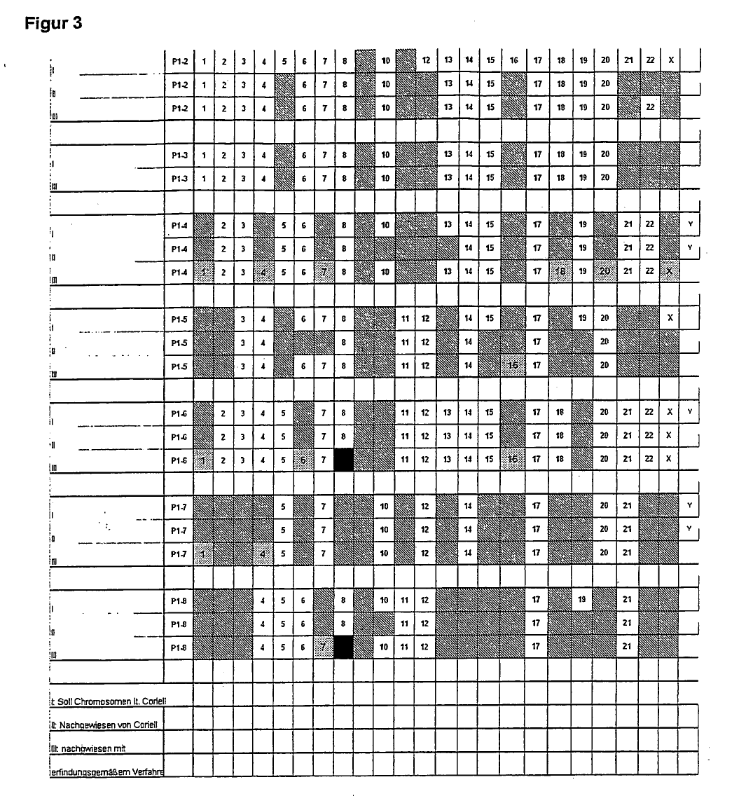

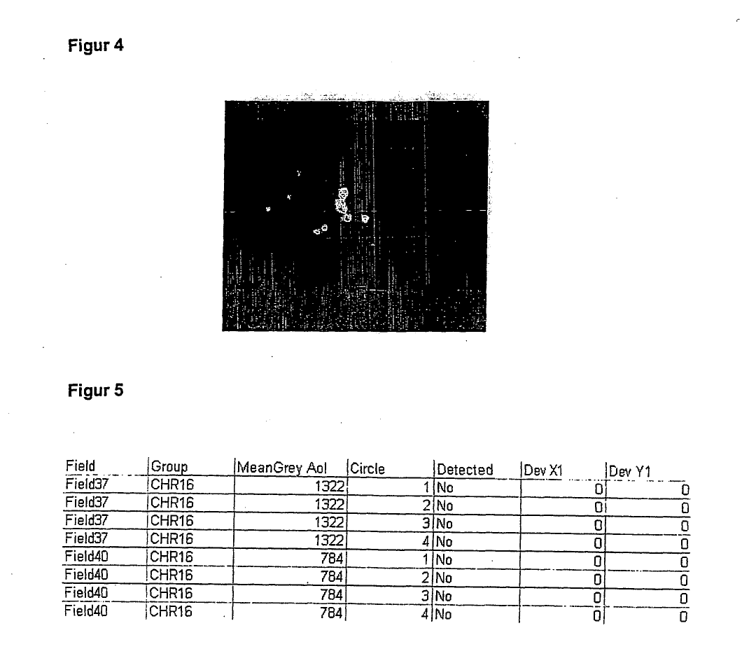

FIG. 4 shows the result of a FISH analysis of a human egg cell with a hybridization kit from the company Vysis.

FIG. 5 shows the image analysis of a scan with the TIFF Analyser program.

The method according to the invention is explained with the aid of the following examples:

### EXAMPLE 1

An investigation is to be conducted to determine whether chromosome 2 is present once or twice in a pole body.

This involved a pole body being washed with distilled water following removal and placed on a coated slide. This pole body formed a sample 1. For comparison, a sample 2 with two pole bodies was prepared in the same way.

### Single Cell WGA-PCR

With a single cell WGA-PCR the two samples were amplified. A single cell WGA-PCR is designed to amplify the genetic material of a single cell or a small number of cells. The single cell WGA-PCR is carried out on a slide, whereby

1 μl PCR mix and 5 μl mineral oil were added to each of the samples.
25 μl PCR mix have the following constituents:

| | |
|---|---|
| 19.125 μl | ampoule water |
| 2.5 μl | MgCl₂ (25 mM) |
| 2.5 μl | dNTP mix (per 2 mM) |
| 0.375 μl | HotStar Taq DNA polymerase from |
| | Qiagen |
| | (5 U/μl) |
| 0.5 μl | Alel primer (100 pmol/μl) |

The Ale1 primer has the following sequence:

| | | |
|---|---|---|
| Ale1 | 5′-TCCCAAAGTGCTGGGATTACAG-3′ | (SEQ ID No. 1) |

The PCR preparations, each consisting of one sample, the PCR mix and the oil film were cycled under the following PCR conditions:

| | |
|---|---|
| Denaturation: | 15 min at 95° C. |
| 40 cycles | 30 sec at 94° C. |
| | 30 sec at 62° C. |
| | 30 sec at 72° C. |
| Elongation | 10 min at 72° C. |

With this PCR, several different sections of the samples are amplified simultaneously. They can therefore also be referred to as WGA-PCR.

The PCR product was transferred into 20 μl TE buffer. 2 μl of this were analysed on a polyacrylamide gel, 15 μl were amplified with a marker PCR. The remainder was frozen at −20° C.

### Marker PCR

The marker PCR was designed to detect whether given sections of the samples had been amplified with the single cell WGA-PCR.

Using the marker PCR, parts of the PCR product of the single cell PCR were amplified with another primer pair in each case, which are each specific for one of these sections. The following PCR preparation was mixed for each single marker PCR:

| | |
|---|---|
| 1.5925 μl | ampoule water |
| 0.6 μl | buffer |
| 0.6 μl | MgCl₂ (25 mM) |
| 0.0325 μl | Taq polymerase (5 U/μl) from |
| | Promega |
| 0.075 μl | PCR product of single cell PCR |
| 2.5 μl | primer (100 pmol/μl) presented |

The primer pairs were presented in reaction vessels of microtitre plates and the remaining PCR preparation was pipetted onto them. To detect the sections amplified by chromosome 2 in the single cell PCR, the following eight primer pairs were used:

| | |
|---|---|
| RH102790 | 5′-TGAAGTCATCGTCTATAAGGCA-3′ |
| | 5′-TCTATTTGTCCTGGGACCCA-3′ |
| SHGC-31419 | 5′-TCCTATTTTGAGGGCGAGG-3′ |
| | 5′-ATAAATACAAACATGTCAGACTGGG-3′ |
| SHGC-62010 | 5′-AAGGTTTTATAATGGAAACACTG-3′ |
| | 5′-TGAGTTCTGGAATTCATTACATA-3′ |
| RH102813 | 5′-CCAACCACTTCAAGAAATAGGC-3′ |
| | 5′-AATACAGTGTGGCCAAAGCC-3′ |
| SHGC-30955 | 5′-GTTTTTTCTTTGAGTGACACAAGC-3′ |
| | 5′-ACTTGTGTGATTTGTAAGCTGAAAC-3′ |
| G62066 | 5′-GCCTCACAAGCCTCATCAGT-3′ |
| | 5′-CGGACTTGTCTAGAAATGAGCA-3′ |
| G31877 | 5′-TTGGCCTCCACTTTACAGAC-3′ |
| | 5′ CACCCGGCCTATGGACAGA-3′ |
| SHGC-144725 | 5′-ATGGACAGGATGGTGATAAGGAA-3′ |
| | 5′-AGATGCAAGGAAAGATGCTTACG-3′ |

The sequences of the above primers are depicted in SEQ ID no. 2-17.

With the following PCR conditions, the two samples were each amplified in eight PCR preparations each with one of the primer pairs mentioned above:

| | |
|---|---|
| Denaturation: | 3 min at 95° C. |
| 35 cycles | 30 sec at 95° C. |
| | 30 sec at 55° C. |
| | 30 sec at 72° C. |
| Elongation | 10 min at 72° C. |

Following amplification, the 16 amplified products were each analysed with a loading buffer on a polyacrylamide gel to see whether the given sequence section in each case was present, i.e. whether the amplification had been positive or negative. The corresponding illustrations of the electrophoresis investigation on polyacrylamide gel are shown in FIGS. 2a and 2b, wherein FIG. 2a shows the bands of sample 1 and FIG. 2b the bands of sample 2. With the aid of these illustrations, it is possible to identify that with sample 1 two positive amplified products have been determined. The remaining six other amplified products are negative, i.e. only two of the sections of chromosome 2 predetermined by the choice of primers of the marker PCR have been amplified with the single cell PCR. With sample 2, eight positive amplified products were determined, i.e. all eight given sections had been amplified with the single cell PCR. The results are summarised in FIG. 1.

With the example shown in FIGS. 2a and 2b, it can clearly be seen that in the case of sample 2 all eight sections have been amplified, whereas in the case of sample 1 only the sections numbered 2 and 7 have been amplified, while the signal for the section identified as number 2 is weaker. It is advisable in principle for a threshold value to be established with which a positive amplification of a section is discriminated from a negative amplification, in order to obtain a purely digital outcome, which can also be depicted by “0” for a negative amplification and “1” for a positive amplification, for example. These threshold values must be empirically defined, depending on the method chosen for detecting the sections.

Example 1 shows very strikingly the effect that with a smaller number of given sequences (here: chromosome 2 in sample 1) in a sample, fewer sections of the sequence are amplified than with a higher number of given sequences (here chromosome 2 in sample 2) in a sample.

Whether this result is based on a purely random sample or has some significance can be determined using statistical methods. A suitable statistical method is the χ² test (also: Chi-square test), as described in e.g. L Cavalli—Sforza, Biometrie, Gustav Fischer Verlag Stuttgart, 1974 in chapter 22. When using this test on the results obtained, a value for χ² of 9.6 and an error probability P of 0.003 are produced. This means that the hypothesis “differences in the observed abundances are random” is rejected with an error probability of P 0.003.

It was therefore established using this method that more chromosomes 2 are contained in sample 2 than in sample 1.

If the method described above is carried out several times and the results evaluated statistically, the absolute number of the chromosome 2 in a sample can be determined on the basis of the statistical data thereby obtained by means of the abundance of the existence or otherwise of the given sections in the amplified product. This represents a validation of the method for counting the absolute number of given sequences in a sample. The influence of the threshold values described above must be considered in connection with this validation. If the threshold value is set high, there are fewer positive amplifications of the sections, whereas with a low threshold value there are more positive amplifications.

### EXAMPLE 2

7 cell lines (P1-2 to P1-8) were tested for the presence or otherwise of given chromosomes. The cell lines were obtained from Coriell. The cells obtained from Coriell had already been tested by Coriell itself for the presence or otherwise of given chromosomes. The cells were also tested using the method in the invention. The result is depicted in FIG. 3.

The cells' DNA is delivered and contains, according to the packing leaflet, a given panel of human chromosomes. In addition to this statement from Coriell, a test result can still be obtained from Coriell's website, based on a blotting test. It is unclear why the company provides two sets of details. The blotting test is clearly sensitive enough also to detect chromosomes that are only contained in a fraction of the cells. The third line in FIG. 3 shows the result of the chip in each case.

### Result:

In over 90% of cases the results of the method according to the invention agree with those of the other methods.

Experimental implementation of the PCR on Coriell cells in accordance with the present invention:

10 ng chromosomal DNA were introduced into a 25 μl Ale PCR (whole genome amplification with the primer Ale1; see Example 1) and cycled under standard conditions:

| Ampuwa (Fresenius) | x times quantity |
|---|---|
| Buffer (10×), 15 mM MgCl₂ | 25 |
| (Qiagen) | [μl] |
| dNTPs (2 mM) (Abgene) | 18.125 |
| Hot Start Taq polymerase | 2.5 |
| (5 U/μl) Qiagen | 2.5 |
| Ale primer no. 813 (100 pmol/μl) | 0.375 |
| | 0.5 |
| | 24.0 |
| Positive control DNA | [μl] |
| (10 ng/μl) | 1 |
| | 25.0 |

| | Temperature | Time |
|---|---|---|
| 1: Initial denaturation | 95° C. | 15 min |
| 2: Denaturation | 94° C. | 30 s |
| 3: Annealing | 62° C. | 30 s |
| 4: Extension | 72° C. | 30 s |
| 5: Final extension | 72° C. | 10 min |
| 6: Holding temperature | 8° C. | ∞ |
| Number of cycles: Step 2-4 | 40 | |

The 25 μl PCR preparation was purified (PCR Purification Kit, Macherey & Nagel) and added to 250 μl elution buffer. 1 μl of this was added to each anchor of a chip presented with Master Mix.

| | per spot |
|---|---|
| | (1 μl) |
| Ampuwa (Fresenius) | 0.705 |
| Buffer (10x), 15 mM MgCl₂ | 0.1 |
| (Qiagen) | |
| dNTPs (2 mM) (Abgene) | 0.1 |
| Hot Start Taq polymerase | 0.015 |
| (5 U/μl) Qiagen | |
| Total volume | 0.92 |
| Primer pair (per 10 pmol/μl) | 0.08 |
| Total volume | 1.00 |

and this was cycled and hybridized under the following conditions.

| | Temperature | Time |
|---|---|---|
| 1: Initial denaturation | 95° C. | 15 min |
| 2: Denaturation | 94° C. | 30 s |
| 3: Annealing | 62° C. | 30 s |
| 4: Extension | 72° C. | 30 s |
| 5: Final extension | 72° C. | 10 min |
| 6: Hybridisation | 40° C. | 30 min |
| Number of cycles: Step 2-4 | 40 | |

The slides were then rinsed and scanned in (standard scanner made by Axxon or Tecan).

### EXAMPLE 3

During the reduction division of a human egg cell, the diploid chromosome set with 4 copies of a sequence is reduced to the mature egg cell with only one copy. The division takes place in 2 stages:

a. Division of the homologous chromosomes in the egg cell←→1^{st} pole body
b. Division of the chromatides in the mature egg cell←→2^{nd} pole body

The first pole body contains 2 copies of a sequence, the mature egg cell and the second pole body each contain one copy of a sequence.

The following distributions (in some cases, wrong distributions) are conceivable:

Mature egg cell contains 4 copies←→pole bodies contain no copies
Mature egg cell contains 3 copies←→pole bodies contain one copy
Mature egg cell contains 2 copies←→pole bodies contain 2 copies
Mature egg cell contains 1 copy←→pole bodies contain 3 copies
Mature egg cell contains no copies←→pole bodies contain 4 copies
Wrong distributions occur and can be used to demonstrate the accuracy of the inventive method.

In the example, corresponding pole bodies and egg cells are investigated. If the fluorescence in situ hybridization (FISH) shows 4 correct signals, no sequence may be detected in the pole bodies. If the FISH shows 3 signals or fewer, the inventive method must be positive (the pole bodies contain at least one copy).

The following experiment shows the correspondence of the chip results to an established FISH method. The single cell processing and FISH hybridization take place according to the Vysis protocol, which accompanies each kit.

The result of the FISH analysis of a human egg cell with a hybridization kit from Vysis is illustrated in FIG. 4. The largest points (blue in the original) are fluorescence-marked probe molecules, which specifically detect chromosome 16. 4 positive signals (no artifacts) indicate that 4 chromatides are located in the egg cell; during meiosis the chromatides wrongly remained in the egg cell.

### Corresponding Analysis Per Chip:

The pole body amplified product is investigated following whole genome amplification for the presence or otherwise of all chromosomes. The experimental procedure is described above in Examples 1 and 2. The PCR conditions and components are as in Example 2, however the template (DNA) is replaced with a pole body, which is located on the chip as a template.

### Result of the Scan:

The image analysis of the picture file is undertaken using the TIFFAnalyzer program from Alopex. Chromosome 16 does not have to be detected. The result of this analysis is illustrated in FIG. 5, column 5.

Accordingly, the corresponding first body contains no chromosome 16. This can be shown by the chip.

1. A method for determining the abundance of a given sequence or several sequences identical or nearly identical to the given sequence in a sample, comprising the following steps:
carrying out of one or more amplification reactions by means of which several different target sections of the sequence or sequences of the sample can be amplified to give an amplified product,
detection of whether given different target sections of the sequence of the sample have been amplified and
determination of the abundance of the sequence(s) in the sample by means of the abundance of the presence or otherwise of the given different target sections in the amplified product.

2. The method according to claim 1 for determining the abundance n of a given sequence or several sequences identical or nearly identical to the given sequence in a sample, comprising the steps:
(a) providing a sample containing the sequence in an abundance n to be determined;
(b) providing primers with which a number m of target sections of the given sequence in the sample can be amplified into different amplified products h each case;
(c) carrying out one or more amplification reactions using the sample from (a) and the primers from (b), in which the reaction conditions are chosen such that the number of successful amplification reactions depends on the abundance n of the given sequence in the sample;
(d) detecting the amplified target sections from step (c) and determining the number of successful amplification reactions;
(e) determining the abundance n of the given sequence contained in the sample.

3. The method according to claim 1, further comprising the step:
(f) determining the abundance n of the given sequence contained in the sample by comparison with one or several controls, in which a known abundance of the given sequence is present.

4. The method according to claim 3,
wherein the controls from step (f) are control samples, which contain the sequence in a known abundance, and wherein the control samples are subject to the same amplification conditions as the sample.

5. The method according to claim 3,
wherein the controls from step (f) are validated data from control samples, which contain the sequence in a known abundance and are subject to the same amplification conditions as the sample.

6. The method according to claim 1,
wherein carrying out the amplification reaction from step (b) comprises the following steps:
(i) carrying out a first amplification reaction with one or more non-specific primers, which amplify the given sequence non-specifically,
(ii) carrying out a second amplification reaction with primers specific to the respective target sections.

7. The method according to claim 1, wherein the sample is a single cell and/or comprises the sequences of a single cell.

8. The method according to claim 1, wherein the sample is a pole body and/or comprises the sequences of a single pole body.

9. The method according to claim 1, wherein the given sequence is a chromosome or a fragment or part thereof.

10. The method according to claim 1, wherein the abundance n to be determined of the given sequence in the sample lies between 0 and 100, preferably within the range 0 to 30, preferably within the range 0 to 10.

11. The method according to claim 10, wherein the abundance n to be determined of the given sequence in the sample lies between 0 and 5.

12. The method according to claim 11, wherein the abundance n to be determined of the given sequence in the sample is 0, 1, 2 or 3.

13. The method according to claim 1, wherein a threshold value is established for a successful amplification reaction.

14. The method according to claim 1, wherein the reaction conditions are chosen such that the efficiency of the amplification reaction for n=1 is between 0.2 and <1 for a given target section.

15. The method according to claim 10, wherein the reaction conditions are chosen such that the efficiency of the amplification reaction for n=1 is between 0.4 and 0.6, preferably around 0.5, for a given target section.

16. The method according to claim 1, wherein the number m of specific target sections for the given sequence is at least 4.

17. The method according to claim 16, wherein the number m of specific target sections for the given sequence is at least 6.

18. The method according to claim 16, wherein the number m of specific target sections for the given sequence is at least 8.

19. The method according to claim 1, wherein when determining the abundance n of the given sequence(s) in the sample by means of the presence or otherwise of amplified products of the given different target sections validated data are used, wherein the validated data have been obtained from control samples in which a known abundance of the given sequence is present, so that the absolute abundance n of the given sequences is determined.

20. The method according to claim 1, wherein a type of primer (statistical primer) is used to carry out the amplification reaction, which is suited to amplifying different target sections of the sequence(s).

21. The method according to claim 20,
wherein in order to investigate whether given target sections of the given sequence have been amplified, the amplified product is amplified by means of several types of primers, each of which is specific to one or more of the given different target sections.

22. The method according to claim 1, wherein several types of primers are used to carry out the amplification reactions in the sample, which are specific to one or more of the given different target sections.

23. The method according to claim 1, wherein the amplified product is analysed by means of electrophoresis, hybridization analysis on a DNA array, a marking method, a bead system or another optical, electrical or electrochemical measurement for the presence of the different sections, wherein it is determined whether the quantity of amplified product of a particular target section exceeds a given threshold.

24. The method according to claim 1, wherein specific primers with markings are used and during the investigation of the given different target sections it is detected whether the marking assigned to a given different target section by means of the respective primer exceeds a given threshold value.

25. The method according to claim 1, wherein the abundance of the given sequence(s) is determined from the abundance of the given target sections in the amplified product by means of statistical analysis.

26. The method according to claim 25, wherein the statement of the statistical analysis is subject to an error probability of under 10% and preferably under 1%.

27. A kit for carrying out the method according to claim 1, comprising
(i) one or more specific primers with which a number m of target sections of the given sequence, the abundance of which is to be determined in a sample, can each be amplified into different amplified products,
(ii) if appropriate, control samples for each possible value n for the abundance of the given sequence in the control sample and/or
(iii) if appropriate, results of amplification reactions with the primers from (i) and/or of control samples with the abundance of the sequence to be counted known,
(iv) details of the reaction conditions for the amplification reactions.

28. A kit according to claim 27, further comprising:
(v) one or more non-specific primers, with which the given sequence can be amplified non-specifically.
(vi) if appropriate, results of amplification reactions with the primers from (i) and (v) and/or of control samples with the abundance of the sequence to be counted known,
(vii) details of the reaction conditions for the amplification reactions.

29. The kit according to claim 27, further comprising reagents for carrying out an amplification reaction.

30. The kit according to claim 27, further comprising a solid carrier for carrying out the amplification reaction and/or detecting the amplified target sections.

31. The kit according to claim 30, wherein the solid carrier is a chip or a slide.

32. The kit according to claim 27, further comprising suitable probes for detecting the specific target sections.

33. Apparatus for carrying out a method according to claim 1, wherein the apparatus comprises:
(a) a solid carrier on which the method is carried out,
(b) a mechanism for detecting the amplified products on the solid carrier from (a) and also
(c) either stored control data, which were obtained from control samples, in which the given sequence is present in a known abundance, or
(d) control positioning sites on which control samples can be analysed under the same conditions as the method.
